(19) United States
(12) Patent Application Publication
(10) Pub. No.: US 2024/0122547 A1
(43) Pub. Date: Apr. 18, 2024

(54) HUMAN BODY SIGNAL COLLECTION APPARATUS

(71) Applicant: WUHAN UNITED IMAGING HEALTHCARE SURGICAL TECHNOLOGY CO., LTD., Wuhan (CN)

(72) Inventors: ZHUO-BIAO HE, Wuhan (CN); JUN TIAN, Wuhan (CN); YI-FENG CHEN, Wuhan (CN); DUN ALEX LI, Wuhan (CN)

(21) Appl. No.: 18/275,418

(22) PCT Filed: Aug. 30, 2021
(86) PCT No.: PCT/CN2021/115352

§ 371 Date: Dec. 3, 2023

(30) Foreign Application Priority Data
Feb. 2, 2021 (CN) 202110142469.9

(51) Int. Cl. A61B 5/00; A61B 5/282

(52) U.S. Cl. CPC A61B 5/721; A61B 5/282; A61B 5/6823; A61B 5/7225; A61B 2560/0209; A61B 2560/0214; A61B 2562/0217

(57) ABSTRACT

A human-body-signal collection device (10). The human-body-signal collection device (10) includes a reference electrode (210), a first electrode (220) and a second electrode (230), an adjusting and generating device (30), a first collection device (40), and a first feedback device (50). The first electrode (220) outputs the first signal. The second electrode (230) outputs the second signal. The adjusting and generating device (30) is connected to the reference electrode (210) to output a reference signal to the reference electrode (210). The first collection device (40) collects the first signal and the second signal, and generates a first potential-difference signal based on the first signal and the second signal. The first feedback device (50) is connected to the first collection device (40) and the adjusting and generating device respectively. The first feedback device (50) is configured to collect the reference signal and the first potential-difference signal, and based on which, generate a first feedback signal, and use the first feedback signal to compensate for the first potential-difference signal, so that an influence of an artifact caused by a baseline drift of the first potential-difference signal may be eliminated, thereby reducing the influence of the baseline drift on a detected signal, and improving accuracy of a detected human-body-signal.

### CROSS-REFERENCE TO RELATED APPLICATION

The present application is a U.S. National Stage of International Application No PCT/CN2021/115352, filed on Aug. 30, 2021, which claims priority of Chinese patent application No. 2021101424699, entitled “HUMAN-BODY-SIGNAL COLLECTION DEVICE”, filed on Feb. 2, 2021, which is incorporated herein by reference in its entirety.

### TECHNICAL FIELD

The present application relates to the field of medical technology, in particular to a human-body-signal collection device.

### BACKGROUND

In the process of medical diagnosis, human body signals need to be detected usually by means of electrode-signal detecting instruments. The accuracy of signal detection affects the accuracy of a doctor's diagnosis.

However, in the process of collecting the detected human-body-signal, influenced by aging of an electrode itself, a relative movement between the electrode and skin, and a press on the skin, a baseline drift may occur in the detected human-body-signal. The frequency of an artifact signal caused by the baseline drift will cover the frequency of the detected human-body-signal, thus affecting accuracy of the detection.

### SUMMARY

In view of this, the present application provides a human-body-signal collection device.

The present application provides a human-body-signal collection device, including: a reference electrode, a first electrode and a second electrode, an adjusting and generating device, a first collection device, and a first feedback device. The reference electrode is attached to an area to be detected. The first electrode and the second electrode are attached to a torso of the human body at intervals, the first electrode is configured to output a first signal, and the second electrode is configured to output a second signal. The adjusting and generating device is connected to the reference electrode and configured to output a reference signal to the reference electrode. The first collection device is connected to the first electrode and the second electrode, respectively. The first collection device is configured to collect the first signal and the second signal, and generate a first potential-difference signal according to the first signal and the second signal. The first feedback device is connected to the first collection device and the adjusting and generating device, respectively. The first feedback device is configured to collect the reference signal and the first potential-difference signal, generate a first feedback signal according to the reference signal and the first potential-difference signal, and use the first feedback signal to compensate for the first potential-difference signal, to eliminate an artifact in the first potential-difference signal.

In an embodiment, the first feedback signal includes a first sub-feedback signal. The first feedback device includes a first executing device and a first signal processing device. The first executing device is connected to the first collection device, and configured to receive the first potential-difference signal. The first signal processing device is connected to the adjusting and generating device, the first collection device, and the first executing device, respectively. The first signal processing device is configured to collect the reference signal and the first potential-difference signal, and separate a first reference drift signal, which corresponds to the reference signal, from the first potential-difference signal according to the reference signal. The first signal processing device is further configured to obtain the first sub-feedback signal according to the reference signal and the first reference drift signal, and output the first sub-feedback signal to the first executing device. The first executing device is configured to use the first sub-feedback signal to compensate for the first potential-difference signal, to eliminate a multiplicative artifact in the first potential-difference signal.

In an embodiment, the first feedback signal includes a second sub-feedback signal, and the first feedback device includes a second executing device.

The second executing device is connected to the first executing device and the first signal processing device, respectively. The second executing device is configured to receive the first potential-difference signal, in which the multiplicative artifact has been eliminated. The first signal processing device is further configured to obtain the second sub-feedback signal according to the reference signal and the first reference drift signal, and output the second sub-feedback signal to the second executing device. The second executing device is configured to use the second sub-feedback signal to compensate for the first potential-difference signal in which the multiplicative artifact has been eliminated, to eliminate the additive artifact in the first potential-difference signal.

In an embodiment, the first signal processing device includes a multiplicative-artifact analysis module, an additive-artifact analysis module, and a comprehensive analysis module.

The multiplicative-artifact analysis module is connected to the first collection device and the adjusting and generating device, respectively. The multiplicative-artifact analysis module is configured to, after correlating PN codes of the first potential-difference signal and of the reference signal, analyze a change of an amplitude and a change of a phase of the first potential-difference signal. The change of the amplitude and the change of the phase characterize a change of multiplicative noise.

The additive-artifact analysis module is connected to the multiplicative-artifact analysis module, and configured to demodulate a real part and an imaginary part of an impedance according to the change of the amplitude of the first potential-difference signal.

The comprehensive analysis module is connected to the additive-artifact analysis module, the multiplicative-artifact analysis module, the first executing device, and the second executing device, respectively. In an embodiment, the first collection device further includes a first amplifier, a first gain amplifier, a first filter, and a first analog-to-digital conversion circuit.

A first input terminal of the first amplifier is connected to the first electrode, a second input terminal of the first amplifier is connected to the second electrode. The first amplifier is configured to collect the first signal and the second signal, and generate the first potential-difference signal according to the first signal and the second signal. The first gain amplifier is connected to an output terminal of the first amplifier, and configured to implement a gain for the first potential-difference signal. The first filter is connected to the first gain amplifier, and configured to implement a filtering for the first potential-difference signal, for which the gain has been implemented.

The first analog-to-digital conversion circuit is connected to the first filter, the first executing device, and the first signal processing device, respectively, and configured to implement an analog-to-digital conversion for the first potential-difference signal, for which the filtering has been implemented.

In an embodiment, the first analog-to-digital conversion circuit is connected to the multiplicative-artifact analysis module. The first analog-to-digital conversion circuit is configured to output the first potential-difference signal, for which the analog-to-digital conversion is implemented, to the multiplicative-artifact analysis module.

In an embodiment, the human-body-signal collection device further includes a first sensing device. The first sensing device is connected to the first signal processing device. The first sensing device is configured to detect a first motion signal of the first electrode and output the first motion signal to the first signal processing device, and the first signal processing device is configured to judge whether the first signal processing device works or not according to the first motion signal. In an embodiment, when the first motion signal is greater than a preset value, the first signal processing device works.

In an embodiment, the human-body-signal collection device further includes a first bias adjustment device.

In an embodiment, the human-body-signal collection device further includes a third electrode, a second collection device, and a second feedback device. The third electrode is attached to the torso of the human body, and configured to output a third signal. The second collection device is connected to the second electrode and the third electrode, respectively, and configured to collect the second signal and the third signal, and generate a second potential-difference signal according to the second signal and the third signal.

The second feedback device is connected to the second collection device and the adjusting and generating device, respectively, and configured to collect the reference signal and the second potential-difference signal, and configured to generate a second feedback signal according to the reference signal and the second potential-difference signal, and further configured to use the second feedback signal to compensate for the second potential-difference signal, to eliminate an artifact in the second potential-difference signal. In an embodiment, the human-body-signal collection device further includes a switch. The switch includes a signal input terminal, a first output terminal, and a second output terminal. The signal input terminal is connected to an output terminal of the adjusting and generating device. The first output terminal is connected to the first electrode; and the second output terminal is connected to the third electrode.

In an embodiment, the human-body-signal collection device further includes a central control device. The central control device is connected to the first feedback device and the second feedback device, respectively. The central control device is configured to collect the first potential-difference signal and the second potential-difference signal, in which the artifacts are eliminated, and obtain detected signals based on the first potential-difference signal and the second potential-difference signal.

In an embodiment, the human-body-signal collection device further includes a power supply module. The power supply module includes a battery and a power management module. The battery is connected to the power management module. The power management module is connected to the adjusting and generating device, the first collection device, the first feedback device, the second collection device, and the second feedback device, respectively.

The present application provides a human-body-signal collection device, including a reference electrode, a first electrode and a second electrode, an adjusting and generating device, a first collection device, and a first feedback device. The reference electrode is attached to an area to be detected. The first electrode and the second electrode are attached to a torso of the human body at intervals, the first electrode is configured to output a first signal, and the second electrode is configured to output a second signal. The adjusting and generating device is connected to the reference electrode and configured to output a reference signal to the reference electrode. The first collection device is connected to the first electrode and the second electrode, respectively. The first collection device is configured to collect the first signal and the second signal, and generate a first potential-difference signal according to the first signal and the second signal. The first feedback device is connected to the first collection device and the adjusting and generating device, respectively. The first feedback device is configured to collect the reference signal and the first potential-difference signal, generate a first feedback signal according to the reference signal and the first potential-difference signal, and use the first feedback signal to compensate for the first potential-difference signal, to eliminate an artifact in the first potential-difference signal. The third executing device is connected to the first collection device, to offset capacitance between the human body and a reference ground.

In an embodiment, the reference electrode, the first electrode and the second electrode are wet electrodes.

In an embodiment, the first collection device further includes a first amplifier, a first gain amplifier, a first filter, and a first analog-to-digital conversion circuit.

A first input terminal of the first amplifier is connected to the first electrode, and a second input terminal of the first amplifier is connected to the second electrode. The first amplifier is configured to collect the first signal and the second signal, and generate the first potential-difference signal according to the first signal and the second signal. A first gain amplifier is connected to an output terminal of the first amplifier, and configured to implement a gain for the first potential-difference signal. The first filter is connected to the first gain amplifier, and configured to implement a filtering for the first potential-difference signal, for which the gain has been implemented.

The first analog-to-digital conversion circuit is connected to the first filter, the first executing device, and the first signal processing device, respectively, and configured to implement an analog-to-digital conversion for the first potential-difference signal, for which the filtering has been implemented.

In an embodiment, the first feedback signal includes a third sub-feedback signal. A first terminal of the third executing device is connected between the first electrode and the first input terminal of the first amplifier. A second terminal of the third executing device is connected between the output terminal of the first amplifier and the input terminal of the first gain amplifier, and a control terminal of the third executing device is connected to the first signal processing device. The first signal processing device is further configured to obtain a third sub-feedback signal according to the reference signal and the first reference drift signal, and output the third sub-feedback signal to the control terminal of the third executing device. The control terminal of the third executing device is configured to adjust a capacitance value of the third executing device according to the third sub-feedback signal, to offset the capacitance between the human body and the reference ground.

In an embodiment, the third executing device is connected in parallel with the first amplifier. The third executing device is configured to adjust the capacitance value of the third executing device according to the third sub-feedback signal, which can change impedance of the first collection device, thereby changing a proportion of an input impedance the first collection device, reducing an influence of an equivalent capacitance, and improving anti-interference ability of the detected signal.

In an embodiment, the third executing device is a variable capacitor.

In an embodiment, the human-body-signal collection device further includes a first bias adjustment device connected between the first terminal of the third executing device and the first electrode.

In an embodiment, a signal terminal of the first bias adjustment device is connected to the first feedback device. The first feedback device is configured to generate a voltage adjustment signal according to the reference signal and the first potential-difference signal, and configured to output the voltage adjustment signal to the first bias adjustment device. The first bias adjustment device is configured to adjust a bias voltage according to the voltage adjustment signal, to realize a compensation for a DC bias voltage at the input port of the first collection device.

The human-body-signal collection device provided in the embodiment of the present application includes: the reference electrode, the first electrode and the second electrode, an adjusting and generating device, the first collection device, and the first feedback device. The reference electrode is attached to an area to be detected. The first electrode and the second electrode are attached to the torso of the human body at intervals, the first electrode is configured to output the first signal, and the second electrode is configured to output the second signal. The adjusting and generating device is connected to the reference electrode and configured to output the reference signal to the reference electrode. The first collection device is connected to the first electrode and the second electrode, respectively. The first collection device is configured to collect the first signal and the second signal, and generate the first potential-difference signal according to the first signal and the second signal. The first feedback device is connected to the first collection device and the adjusting and generating device, respectively. The first feedback device is configured to collect the reference signal and the first potential-difference signal, generate the first feedback signal according to the reference signal and the first potential-difference signal, and use the first feedback signal to compensate for the first potential-difference signal, to eliminate an artifact in the first potential-difference signal.

In the human-body-signal collection device, by arranging the adjusting and generating device and the reference electrode, the adjusting and generating device outputs the reference signal to the reference electrode. The superposition of the reference signal and a signal of the to-be-detected area of the human body is outputted to the first feedback device. The first feedback device obtains the first feedback signal based on the reference signal and the first potential-difference signal. The first feedback signal may characterize the baseline change of the reference signal after the reference signal passes through the human body. The first feedback device uses the first feedback signal to compensate for the first potential-difference signal, so that the influence of the artifact caused by the baseline drift of the first potential-difference signal may be eliminated, thereby reducing the influence of the baseline drift on the detected signal, and improving the accuracy of the detected human-body-signal.

### BRIEF DESCRIPTION OF THE DRAWINGS

In order to illustrate the technical solutions of the embodiments of the present application or the prior art more clearly, the drawings needed for the description of the embodiments or the prior art will briefly be introduced. Obviously, the accompanying drawings in the following description are only embodiments of the present application, and those skilled in the art may also obtain other drawings according to the provided drawings without creative efforts.

FIG. 1 is a schematic circuit diagram illustrating a human-body-signal collection device according to an embodiment of the present application;

FIG. 2 is a schematic circuit diagram illustrating the human-body-signal collection device according to another embodiment of the present application;

FIG. 3 is a schematic circuit diagram illustrating the human-body-signal collection device according to another embodiment of the present application.

### REFERENCE SIGNS

human-body-signal collection device 10; reference electrode 210; first electrode 220; second electrode 230; adjusting and generating device 30; first collection device 40; first amplifier 410; first input terminal of first amplifier 411; second input terminal of first amplifier 412; output terminal of first amplifier 413; first gain amplifier 420; first filter 430; first analog-to-digital conversion circuit 440; first feedback device 50; first executing device 510; first signal processing device 520; second executing device 530; third executing device 540; first terminal of third actuator 541; second terminal of third actuator 542; control terminal of third actuator 543; first bias adjustment device 60; first sensing device 610; third electrode 240; second collection device 70; second amplifier 710; first input terminal of second amplifier 711; second input terminal of second amplifier 712; output terminal of second amplifier 713; second gain amplifier 720; second filter 730; second analog-to-digital conversion circuit 740; second feedback device 80; fourth executing device 810; second signal processing device 820; fifth executing device 830; sixth executing device 840; first terminal of sixth executing device 841; second terminal of sixth executing device 842; control terminal of sixth executing device 843; switch 90; signal input terminal 910; first output terminal 920; second output terminal 930; central control device 100; second bias adjustment device 600; second sensing device 620.

### DETAILED DESCRIPTION OF THE EMBODIMENTS

The technical solutions in the embodiments of the application will be clearly and completely described with reference to the drawings of the embodiments of the application. Apparently, the described embodiments are some but not all embodiments of the application. Based on the embodiments in this application, other embodiments may be obtained by those ordinary skill in the art without creative efforts, and these embodiments are all within the scope of protection of this application.

In order to make the objectives, technical solutions, and advantages of the present application clearer and to be better understood, the present application will be further described in detail by means of the following embodiments combining with the accompanying drawings. It should be understood that the specific embodiments described herein are only used to explain the present application, but not intended to limit the present application.

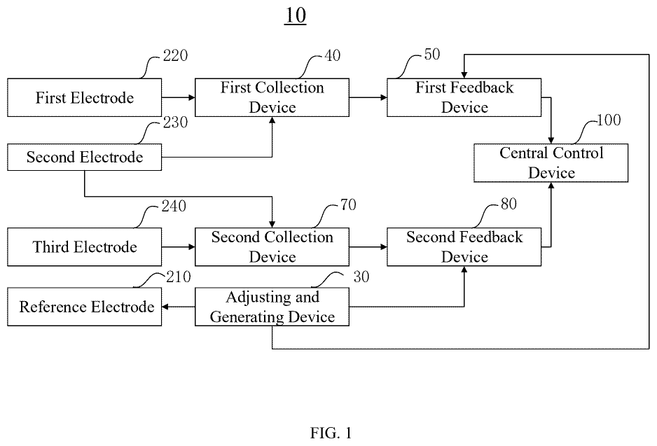

Referring to FIG. 1, the embodiment of the present application provides a human-body-signal collection device 10, including a reference electrode 210, a first electrode 220, a second electrode 230, an adjusting and generating device 30, a first collection device 40, and a first feedback device 50. The reference electrode 210 is attached to an area to be detected. The first electrode 220 and the second electrode 230 are attached to a torso of the human body at intervals. The first electrode 220 is configured to output a first signal. The second electrode 230 is configured to output a second signal. The adjusting and generating device 30 is connected to the reference electrode 210. The adjusting and generating device 30 is configured to output a reference signal to the reference electrode 210. The first collection device 40 is connected to the first electrode 220 and the second electrode 230, respectively. The first collection device 40 is configured to collect the first signal and the second signal, and generate a first potential-difference signal according to the first signal and the second signal. The first feedback device 50 is connected to the first collection device 40 and the adjusting and generating device 30, respectively. The first feedback device 50 is configured to collect the reference signal and the first potential-difference signal, generate a first feedback signal according to the reference signal and the first potential-difference signal, and use the first feedback signal to compensate for the first potential-difference signal, to eliminate an artifact in the first potential-difference signal.

In the human-body-signal collection device 10 provided in the embodiment of the present application, by arranging the adjusting and generating device 30 and the reference electrode 210, the adjusting and generating device 30 outputs the reference signal to the reference electrode 210. A superposition of the reference signal and a signal of the to-be-detected area of the human body is outputted to the first feedback device 50. The first feedback device 50 obtains the first feedback signal based on the reference signal and the first potential-difference signal. The first feedback signal may characterize a baseline change of the reference signal after the reference signal passes through the human body. The first feedback device 50 uses the first feedback signal to compensate for the first potential-difference signal, so that the influence of the artifact caused by the baseline drift of the first potential-difference signal may be eliminated, thereby reducing the influence of the baseline drift on the detected signal, and improving the accuracy of the detected human-body-signal.

The collected human-body-signal may be an electrocardiogram (ECG) signal, an electroencephalogram (EEG) signal, a respiratory signal, a muscle signal, and the like. The area to be detected may be heart, head, skin, muscle, or blood vessel. When the area to be detected is the heart, the human-body-signal collection device 10 is configured to detect cardiomyocytes, and the generated signal is the ECG signal. The ECG signal travels through the human body tissues to positions on the surface of the human body where the electrodes are attached. In an embodiment, the reference electrode 210 is arranged at a position corresponding to the heart. The position where the reference signal is added is approximately the same position where the ECG signal is generated, thereby ensuring that the transmission pathway of the reference signal is consistent with the transmission pathway of the ECG signal, that is, a transmission pathway from the heart to each of the electrodes. After the reference electrode 210 is arranged, the influences of the artifacts caused by motions and breathing, etc., on the ECG signal and the reference signal are approximately identical, therefore the artifact formed in the reference signal may be, to the greatest extent, consistent with the artifact superposed on the ECG.

The reference electrode 210, the first electrode 220, and the second electrode 230 may be wet electrodes. In an embodiment, the reference electrode 210, the first electrode 220 and the second electrode 230 are made of hydrogel materials.

The second electrode 230 is a common electrode, and the first electrode 220 is a detecting electrode. The first signal and the second signal are voltage signals. A difference between the voltage signals detected by the first electrode 220 and the second electrode 230 is the first potential-difference signal. The first potential-difference signal is also a voltage signal. The adjusting and generating device 30 is configured to generate a pseudo random signal. The frequency spectrum of the pseudo random signal is consistent with the frequency spectrums of the ECG signal, etc., and the amplitude of the pseudo random signal is much lower than the amplitudes of the ECG signal, etc., thereby reducing the influences of the first feedback signal on the collection of the ECG signal, and reducing an inaccuracy of a correction, which is caused by a great difference between contact impedances of the skin under different frequencies.

In an embodiment, if y denotes the first potential-difference signal generated by the first collection device 40, and x denotes the first potential-difference signal in an ideal state, then the relationship between y and x may be expressed as y=A×(x+B), where, A denotes a multiplicative artifact impact factor, and B denotes an additive artifact impact factor. The first feedback signal generated by the first feedback device 50 is configured to eliminate the multiplicative artifact impact factor and the additive artifact impact factor.

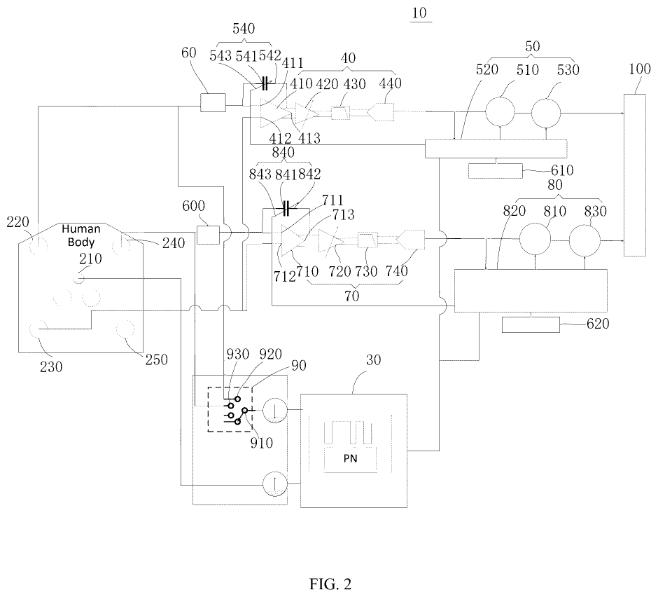

Referring to FIG. 2, in an embodiment, the first feedback signal includes a first sub-feedback signal. The first feedback device 50 includes a first executing device 510 and a first signal processing device 520.

The first executing device 510 is connected to the first collection device 40. The first executing device 510 is configured to receive the first potential-difference signal. The first signal processing device 520 is connected to the adjusting and generating device 30, the first collection device 40, and the first executing device 510, respectively. The first signal processing device 520 is configured to collect the reference signal and the first potential-difference signal, and separate a first reference drift signal, which corresponds to the reference signal, from the first potential-difference signal. The first signal processing device 520 is further configured to obtain the first sub-feedback signal according to the reference signal and the first reference drift signal, and output the first sub-feedback signal to the first executing device 510. The first executing device 510 is configured to use the first sub-feedback signal to compensate for the first potential-difference signal, to eliminate the multiplicative artifact in the first potential-difference signal.

The first sub-feedback signal may offset the multiplicative artifact of the first potential-difference signal. It may be understood that the multiplicative artifact factor may be eliminated by using the first sub-feedback signal to compensate for the first potential-difference signal.

In an embodiment, the first feedback signal includes a second sub-feedback signal, and the first feedback device 50 includes a second executing device 530. The second executing device 530 is connected to the first executing device 510 and the first signal processing device 520, respectively. The second executing device 530 is configured to receive the first potential-difference signal, in which the multiplicative artifact has been eliminated. The first signal processing device 520 is further configured to obtain the second sub-feedback signal according to the reference signal and the first reference drift signal, and output the second sub-feedback signal to the second executing device 530. The second executing device 530 is configured to use the second sub-feedback signal to compensate for the first potential-difference signal in which the multiplicative artifact has been eliminated, to eliminate the additive artifact in the first potential-difference signal.

The second sub-feedback signal may offset the additive artifact in the first potential-difference signal. It may be understood that the additive artifact factor may be eliminated by using the second sub-feedback signal to compensate for the first potential-difference signal.

It may be understood that the first signal processing device 520 simultaneously obtains the first sub-feedback signal and the second sub-feedback signal according to the reference signal and the first reference drift signal.

In an embodiment, the first signal processing device 520 includes a multiplicative-artifact analysis module, an additive-artifact analysis module and a comprehensive analysis module. The multiplicative-artifact analysis module is connected to the first collection device 40 and the adjusting and generating device 30, respectively. The multiplicative-artifact analysis module is configured to, after correlating PN codes of the first potential-difference signal and of the reference signal, analyze changes of the amplitude and the phase of the first potential-difference signal. The changes of the amplitude and phase characterize a change of the multiplicative noise. The additive-artifact analysis module is connected to the multiplicative-artifact analysis module. The additive-artifact analysis module is configured to demodulate a real part and an imaginary part of the impedance according to the change of the amplitude of the first potential-difference signal. The change of the imaginary part is strongly correlated with the additive artifact. The comprehensive analysis module is connected to the additive-artifact analysis module, the multiplicative-artifact analysis module, the first executing device 510, and the second executing device 530, respectively.

In an embodiment, the first collection device 40 further includes a first amplifier 410, a first gain amplifier 420, a first filter 430, and a first analog-to-digital conversion circuit 440. A first input terminal 411 of the first amplifier 410 is connected to the first electrode 220. A second input terminal 412 of the first amplifier 410 is connected to the second electrode 230. The first amplifier 410 is configured to collect the first signal and the second signal, and generate the first potential-difference signal according to the first signal and the second signal. The first gain amplifier 420 is connected to an output terminal 413 of the first amplifier 410. The first gain amplifier 420 is configured to implement a gain for the first potential-difference signal. The first filter 430 is connected to the first gain amplifier 420. The first filter 430 is configured to implement a filtering for the first potential-difference signal, for which the gain has been implemented. The first analog-to-digital conversion circuit 440 is connected to the first filter 430, the first executing device 510, and the first signal processing device 520, respectively. The first analog-to-digital conversion circuit 440 is configured to implement an analog-to-digital conversion for the first potential-difference signal, for which the filtering has been implemented.

The first amplifier 410 is configured to amplify the first signal and the second signal. The first potential-difference signal is a potential difference between the first signal and the second signal. The first gain amplifier 420 is configured to adjust the gain of the first potential-difference signal. The first analog-to-digital conversion circuit 440 is configured to change a type of the first potential-difference signal.

In an embodiment, the first analog-to-digital conversion circuit 440 is connected to the multiplicative-artifact analysis module. The first analog-to-digital conversion circuit 440 is configured to output the first potential-difference signal, for which the analog-to-digital conversion is implemented, to the multiplicative-artifact analysis module.

In an embodiment, the first feedback signal includes a third sub-feedback signal. The human-body-signal collection device 10 further includes a third executing device 540. A first terminal 541 of the third executing device 540 is connected between the first electrode 220 and the first input terminal 411 of the first amplifier 410. A second terminal 542 of the third executing device 540 is connected between the output terminal 413 of the first amplifier 410 and the input terminal of the first gain amplifier 420. A control terminal 543 of the third executing device 540 is connected to the first signal processing device 520. The first signal processing device 520 is further configured to obtain a third sub-feedback signal according to the reference signal and the first reference drift signal, and output the third sub-feedback signal to the control terminal 543 of the third executing device 540. The control terminal 543 of the third executing device 540 is configured to adjust a capacitance value of the third executing device 540 according to the third sub-feedback signal, to offset the capacitance between the human body and the ground.

An equivalent capacitance is formed between the electrodes and the reference ground. The equivalent capacitance causes a reduction of a high-frequency input impedance of the first collection device 40. The reduction of the input impedance worsens the anti-interference ability of the detected signal. The third executing device 540 is connected in parallel with the first amplifier 410. The third executing device 540 adjusts the capacitance value of the third executing device 540 according to the third sub-feedback signal, which can change the impedance of the first collection device 40, thereby changing a proportion of the input impedance of the first collection device 40, reducing the influence of the equivalent capacitance, and improving the anti-interference ability of the detected signal.

In an embodiment, the comprehensive analysis module is further connected to the third executing device 540. The comprehensive analysis module is also configured to import the change of the amplitude and a change of the imaginary part into an optimization algorithm, to obtain the first sub-feedback signal, the second sub-feedback signal, and the third sub-feedback signal. The comprehensive analysis module is further configured to output the third sub-feedback signal to the third executing device 540. The optimization algorithm may be an algorithm such as LMS.

The third executing device 540 may be a variable capacitor (compensating for negative capacitance). The capacitance value of the capacitor is equal to or approximate to the equivalent capacitance, so that an overall input impedance of the human-body-signal collection device 10 is the largest.

For a different detected individual, the equivalent capacitance between the electrodes and the human body is different. Therefore, a compensation method for an initial negative capacitance is used to increase the input impedance of the collection circuit, thereby improving the anti-interference performance. The capacitance value of the variable capacitor may be gradually fixed through multiple experiments. The experimental process includes testing the amplitude of the input signal by means of sweeping frequency, and when the amplitude of the signal in a key frequency band reaches a maximum, the capacitance value of the compensation capacitor is the optimal value.

In an embodiment, the human-body-signal collection device 10 further includes a first bias adjustment device 60. The first bias adjustment device 60 is connected between the first terminal 541 of the third executing device 540 and the first electrode 220.

A stretching of the skin, etc., may cause a change of a DC bias voltage. A polarization voltage (the DC bias voltage) generated by a contact between the electrode and the skin is related to a specific electrode, a position where the electrode is attached to the skin, an attaching time, and a motion, etc. The polarization voltage is as high as +/−300 mV, and the DC voltage affects a common-mode rejection proportion and the input impedance of the first collection device 40, and a compensation is realized by independently adjusting the DC bias voltage at an input port of the first collection device 40.

In an embodiment, a signal terminal of the first bias adjustment device 60 is connected to the first feedback device 50. The first feedback device 50 is configured to generate a voltage adjustment signal according to the reference signal and the first potential-difference signal, and output the voltage adjustment signal to the first bias adjustment device 60. The first bias adjustment device 60 is configured to adjust the bias voltage according to the voltage adjustment signal, to realize the compensation for the DC bias voltage at the input port of the first collection device 40.

In an embodiment, the human-body-signal collection device 10 further includes a first sensing device 610. The first sensing device 610 is arranged proximate to the first electrode 220. The first sensing device 610 is connected to the first signal processing device 520. The first sensing device 610 is configured to detect a first motion signal of the first electrode 220 and output the first motion signal to the first signal processing device 520. The first signal processing device 520 is configured to judge whether it works or not according to the first motion signal.

The first sensing device 610 may be a motion sensing device. The first sensing device 610 may be a speed sensor, an acceleration sensor, or a displacement sensor, etc.

When the first motion signal is greater than a preset value, the first signal processing device 520 works. When the first motion signal is not greater than the preset value, the first signal processing device 520 does not work.

In an embodiment, at an initial period of the startup of the human-body-signal collection device 10, the first signal processing device 520 obtains the third sub-feedback signal according to the reference signal and the first reference drift signal, and the third executing device 540 adjusts the capacitance value according to the third sub-feedback signal. During the detection period of the human-body-signal collection device 10, the capacitance value of the third executing device 540 remains unchanged.

When the first motion signal is greater than the preset value, the first signal processing device 520 obtains the first sub-feedback signal and the second sub-feedback signal according to the reference signal and the first reference drift signal, so that the first executing device 510 and the second executing device 530 perform signal processing according to the feedback signals.

When the first motion signal is not greater than the preset value, the first signal processing device 520 does not process the reference signal and the first reference drift signal. The first executing device 510 and the second executing device 530 do not work.

In an embodiment, the human-body-signal collection device 10 further includes a third electrode 240, a second collection device 70, and a second feedback device 80. The third electrode 240 is attached to the torso of the human body. The third electrode 240 is configured to output a third signal. The second collection device 70 is connected to the second electrode 230 and the third electrode 240, respectively. The second collection device 70 is configured to collect the second signal and the third signal, and generate a second potential-difference signal according to the second signal and the third signal.

The second feedback device 80 is connected to the second collection device 70 and the adjusting and generating device 30, respectively. The second feedback device 80 is configured to collect the reference signal and the second potential-difference signal, and generate a second feedback signal according to the reference signal and the second potential-difference signal. The second feedback device 80 is further configured to use the second feedback signal to compensate for the second potential-difference signal, to eliminate the artifact in the second potential-difference signal.

Usually, the potential difference between the first potential-difference signal and the second potential-difference signal is used as reference information for diagnosing diseases, therefore, during the processing for the first potential-difference signal and for the second potential-difference signal, the related processing may not be performed for the second signal.

In the human-body-signal collection device 10, by arranging the adjusting and generating device 30 and the reference electrode 210, the adjusting and generating device 30 outputs the reference signal to the reference electrode 210. A superposition of the reference signal and the signal of the to-be-detected area of the human body is outputted to the second feedback device 80. The second feedback device 80 obtains the second feedback signal based on the reference signal and the second potential-difference signal. The second feedback signal may characterize the baseline change of the reference signal after the reference signal passes through the human body. The second feedback device 80 uses the second feedback signal to compensate for the second potential-difference signal, so that the influence of the artifact caused by the baseline drift of the second potential-difference signal may be eliminated, thereby reducing the influence of the baseline drift on the detected signal, and improving the accuracy of the detected human-body-signal.

In an embodiment, the human-body-signal collection device 10 may further include other detecting electrodes.

In an embodiment, the second feedback signal includes a fourth sub-feedback signal, and the second feedback device 80 includes a fourth executing device 810 and a second signal processing device 820.

The fourth executing device 810 is connected to the second collection device 70, and the fourth executing device 810 is configured to receive the second potential-difference signal.

The second signal processing device 820 is connected to the adjusting and generating device 30, the second collection device 70, and the fourth executing device 810. The second signal processing device 820 is configured to collect the reference signal and the second potential-difference signal, and separate a second reference, which corresponds to the reference signal, from the second potential-difference signal. The second signal processing device 820 is further configured to obtain the fourth sub-feedback signal according to the reference signal and the second reference drift signal, and output the fourth sub-feedback signal to the fourth executing device 810. The fourth executing device 810 is configured to use the fourth sub-feedback signal to compensate for the second potential-difference signal, to eliminate the multiplicative artifact in the second potential-difference signal.

The fourth sub-feedback signal may offset the multiplicative artifact of the second potential-difference signal. It may be understood that the multiplicative artifact factor may be eliminated by using the fourth sub-feedback signal to compensate for the second potential-difference signal.

In an embodiment, the second feedback signal includes a fifth sub-feedback signal. The second feedback device 80 includes a fifth executing device 830.

The fifth executing device 830 is connected to the fourth executing device 810 and the second signal processing device 820, respectively. The fifth executing device 830 is configured to receive the second potential-difference signal, in which the multiplicative artifact has been eliminated. The second signal processing device 820 is further configured to obtain the fifth sub-feedback signal according to the reference signal and the second reference drift signal, and output the fifth sub-feedback signal to the fifth executing device 830. The fifth executing device 830 is configured to use the fifth sub-feedback signal to compensate for the second potential-difference signal, in which the multiplicative artifact has been eliminated, to eliminate the additive artifact in the second potential-difference signal.

The fifth sub-feedback signal may offset the additive artifact in the second potential-difference signal. It may be understood that the additive artifact factor may be eliminated by using the fifth sub-feedback signal to compensate for the second potential-difference signal.

In an embodiment, the second collection device 70 further includes a second amplifier 710, a second gain amplifier 720, a second filter 730, and a second analog-to-digital conversion circuit 740. A first terminal 711 of the second amplifier 710 is connected to the third electrode 240. A second input terminal 712 of the second amplifier 710 is connected to the second electrode 230. The second amplifier 710 is configured to collect the third signal and the second signal, and generate the second potential-difference signal according to the third signal and the second signal. The second gain amplifier 720 is connected to an output terminal 713 of the second amplifier 710, and the second gain amplifier 720 is configured to implement a gain for the second potential-difference signal. The second filter 730 is connected to the second gain amplifier 720. The second filter 730 is configured to implement a filtering for the second potential-difference signal, for which the gain has been implemented. The second analog-to-digital conversion circuit 740 is connected to the second filter 730, the fourth executing device 810, and the second signal processing device 820, respectively. The second analog-to-digital conversion circuit 740 is configured to implement an analog-to-digital conversion for the second potential-difference signal, for which the filtering has been implemented.

In an embodiment, the second feedback signal includes a sixth sub-feedback signal. A sixth executing device 840 is further included. A first terminal 841 of the sixth executing device 840 is connected between the third electrode 240 and the first terminal 711 of the second amplifier 710. A second terminal 842 of the sixth executing device 840 is connected between the output terminal 713 of the second amplifier 710 and the input terminal of the second gain amplifier 720. The control terminal 843 of the sixth executing device 840 is connected to the second signal processing device 820. The second signal processing device 820 is further configured to obtain the sixth sub-feedback signal according to the reference signal and the second reference drift signal, and output the sixth sub-feedback signal to the control terminal 843 of the sixth executing device 840. The control terminal 843 of the sixth executing device 840 is configured to adjust a capacitance value of the sixth executing device 840 according to the sixth sub-feedback signal, to offset the capacitance between the human body and the ground.

The sixth executing device 840 is connected in parallel with the second amplifier 710. The sixth executing device 840 adjusts the capacitance value of the sixth executing device 840 according to the sixth sub-feedback signal, which can change the impedance of the second collection device 70, thereby changing the proportion of the input impedance of the second collection device 40, reducing the influence of the equivalent capacitance, and improving the anti-interference ability of the detected signal.

The sixth executing device 840 may be a variable capacitor (compensating for negative capacitance). The capacitance value of the capacitor is equal to or approximate to the equivalent capacitance, so that an overall input impedance of the human-body-signal collection device 10 is the largest.

In an embodiment, the human-body-signal collection device 10 further includes a second bias adjustment device 600. The second bias adjustment device 600 is connected between the first terminal 841 of the sixth executing device 840 and the third electrode 240. A compensation is realized by independently adjusting the DC bias voltage at an input port of the second collection device 70.

In an embodiment, the human-body-signal collection device 10 further includes a second sensing device 620. The second sensing device 620 is arranged proximate to the third electrode 240. The second sensing device 620 is connected to the second signal processing device 820. The second sensing device 620 is configured to detect a second motion signal of the third electrode 240, and output the second motion signal to the second signal processing device 820. The second signal processing device 820 is configured to judge whether it works or not according to the second motion signal.

In an embodiment, the human-body-signal collection device 10 further includes a switch 90. The switch 90 includes a signal input terminal 910, a first output terminal 920, and a second output terminal 930. The signal input terminal 910 is connected to the output terminal of the adjusting and generating device 30. The first output terminal 920 is connected to the first electrode 220. The second output terminal 930 is connected to the third electrode 240.

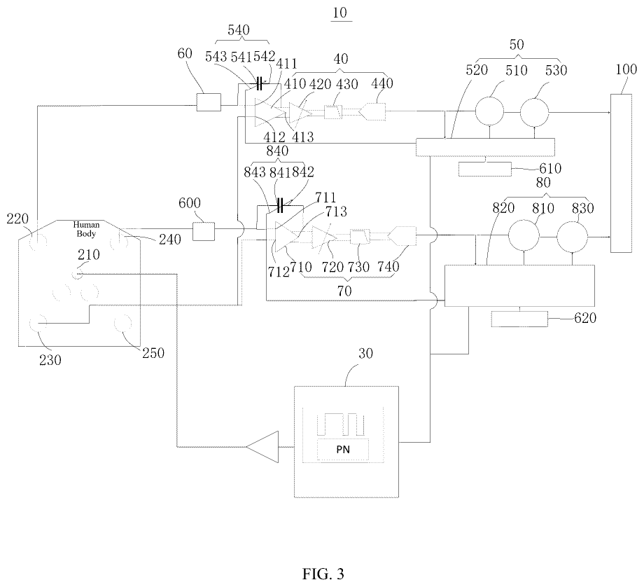

Referring to FIG. 3, if the human-body-signal collection device 10 is a current detection device, when the first output terminal 920 is connected to the signal input terminal 910, the first electrode 220 is connected to the adjusting and generating device 30 through the first output terminal 920. The adjusting and generating device 30, the reference electrode 210, the human body, and the first electrode 220 (a detecting electrode) form a closed loop. When the second output terminal 930 is connected to the signal input terminal 910, the third electrode 240 is connected to the adjusting and generating device 30 through the second output terminal 930. The adjusting and generating device 30, the reference electrode 210, the human body, and the third electrode 240 (a detecting electrode) form a closed loop. By switching the switch 90 to connect different electrodes for detection, it can be ensured that reference signals are consistent.

If the human-body-signal collection device 10 is a voltage detection device, there is no need to form a closed loop, what are needed are that the first collection device 40 needs to obtain an output voltage of the first electrode 220, and that the second collection device 70 needs to obtain an output voltage of the third electrode 240.

In an embodiment, the human-body-signal collection device 10 further includes a central control device 100. The central control device 100 is connected to the first feedback device 50 and the second feedback device 80, respectively. The central control device 100 is configured to collect the first potential-difference signal and the second potential-difference signal, in which the artifacts are eliminated, and obtain detected signals based on the first potential-difference signal and the second potential-difference signal.

In an embodiment, the human-body-signal collection device 10 further includes a power supply module. The power supply module includes a battery and a power management module. The battery is connected to the power management module. The power management module is connected to the adjusting and generating device 30, the first collection device 40, the first feedback device 50, the second collection device 70, and the second feedback device 80, respectively.

The battery may be a lithium battery or a battery having medium of any other chemical element. A rechargeable battery, or a battery of any other type, may be used to supply power. The battery is charged in a wired or wireless manner used by the power management module. The power management module may also record the power of the battery, and may stop supplying power when the power is too low, thereby ensuring a normal operation of the system.

In an embodiment, the human-body-signal collection device 10 further includes a clock. The clock is connected to the adjusting and generating device 30, the first collection device 40, the first feedback device 50, the second collection device 70, and the second feedback device 80, respectively. The clock is configured to regulate the working timing of the adjusting and generating device 30, of the first collection device 40, of the first feedback device 50, of the second collection device 70, and of the second feedback device 80.

All technical features in the embodiments above may be combined arbitrarily, and for the sake of conciseness of the description, all possible combinations of various technical features in the embodiments above have not been described. However, as long as there is no contradiction in the combinations of these technical features, these combinations should all be considered to be within the scope of the present description.

The embodiments above are several implementation modes of the present application, and the description of the embodiments is relatively specific and detailed, but should not be understood to limit the scope of the present application. It should be noted that, for the ordinary skilled in the art, various modifications and improvements may be made without departing from the concepts of the present application, and all these modifications and improvements are all within the protection scope of the present application. Therefore, the scope of protection of the patent application should be based on the appended claims.

Finally, it should also be noted that in the present disclosure, relational terms, such as “first” and “second” etc., are only used to distinguish one entity or operation from another, but do not necessarily require or imply that there are any such actual relationship or order for these entities or operations. Furthermore, the terms “include”, “comprise”, or any other variation thereof are intended to cover a non-exclusive inclusion, such that a process, a method, an article, or an apparatus including a series of elements not only includes those elements, but also includes elements not expressly listed, or includes other elements inherent in the process, in the method, in the article, or in the device. Without further limitations, an element defined by the phrase “including a . . . ” does not exclude the presence of additional identical elements included in the process, in the method, in the article, or in the device.

Embodiments in the description are described in a progressive manner, and the description of each embodiment focuses on the difference of the embodiment from other embodiments, and the same and similar parts of all embodiments may be referred to each other.

The above description of the embodiments is disclosed to enable those skilled in the art to implement or use the present application. Various modifications of these embodiments will be obvious for those skilled in the art, and the general principles defined herein may be implemented in other embodiments without departing from the spirits or the scope of the present application. Therefore, the present application will not be limited to the embodiments shown herein, but has the widest scope consistent with the principles and novel features disclosed herein.

1. A human-body-signal collection device, comprising:
a reference electrode, attached to an area to be detected;
a first electrode and a second electrode, wherein the first electrode and the second electrode are attached to a torso of the human body at intervals, the first electrode is configured to output a first signal, and the second electrode is configured to output a second signal;
an adjusting and generating device, connected to the reference electrode, and configured to output a reference signal to the reference electrode;
a first collection device, connected to the first electrode and the second electrode, respectively, wherein the first collection device is configured to collect the first signal and the second signal, and generate a first potential-difference signal according to the first signal and the second signal; and
a first feedback device, connected to the first collection device and the adjusting and generating device, respectively, wherein the first feedback device is configured to collect the reference signal and the first potential-difference signal, generate a first feedback signal according to the reference signal and the first potential-difference signal, and use the first feedback signal to compensate for the first potential-difference signal.

2. The human-body-signal collection device according to claim 1, wherein:
the first feedback signal comprises a first sub-feedback signal, and the first feedback device comprises:
a first executing device, connected to the first collection device, and configured to receive the first potential-difference signal; and
a first signal processing device, connected to the adjusting and generating device, the first collection device, and the first executing device, respectively, wherein the first signal processing device is configured to collect the reference signal and the first potential-difference signal, and separate a first reference drift signal, which corresponds to the reference signal, from the first potential-difference signal according to the reference signal; the first signal processing device is further configured to obtain the first sub-feedback signal according to the reference signal and the first reference drift signal, and output the first sub-feedback signal to the first executing device; and the first executing device is configured to use the first sub-feedback signal to compensate for the first potential-difference signal.

3. The human-body-signal collection device according to claim 2, wherein:
the first feedback signal comprises a second sub-feedback signal;
the first feedback device comprises a second executing device;
the second executing device is connected to the first executing device and the first signal processing device, respectively; the second executing device is configured to receive the first potential-difference signal, in which a multiplicative artifact has been eliminated; the first signal processing device is further configured to obtain the second sub-feedback signal according to the reference signal and the first reference drift signal, and output the second sub-feedback signal to the second executing device; the second executing device is configured to use the second sub-feedback signal to compensate for the first potential-difference signal in which the multiplicative artifact has been eliminated.

4. The human-body-signal collection device according to claim 3, wherein the first signal processing device comprises:
a multiplicative-artifact analysis module, connected to the first collection device and the adjusting and generating device, respectively, wherein the multiplicative-artifact analysis module is configured to, after correlating PN codes of the first potential-difference signal and of the reference signal, analyze a change of an amplitude and a change of a phase of the first potential-difference signal, and the change of the amplitude and the change of the phase characterize a change of multiplicative noise;
an additive-artifact analysis module, connected to the multiplicative-artifact analysis module, and configured to demodulate a real part and an imaginary part of an impedance according to the change of the amplitude of the first potential-difference signal; and
a comprehensive analysis module, connected to the additive-artifact analysis module, the multiplicative-artifact analysis module, the first executing device, and the second executing device, respectively.

5. The human-body-signal collection device according to claim 4, wherein the first collection device further comprises:
a first amplifier, wherein a first input terminal of the first amplifier is connected to the first electrode, a second input terminal of the first amplifier is connected to the second electrode, and the first amplifier is configured to collect the first signal and the second signal, and generate the first potential-difference signal according to the first signal and the second signal;
a first gain amplifier, connected to an output terminal of the first amplifier, and configured to implement a gain for the first potential-difference signal;
a first filter, connected to the first gain amplifier, and configured to implement a filtering for the first potential-difference signal, for which the gain has been implemented; and
a first analog-to-digital conversion circuit, connected to the first filter, the first executing device, and the first signal processing device, respectively, and configured to implement an analog-to-digital conversion for the first potential-difference signal, for which the filtering has been implemented.

6. The human-body-signal collection device according to claim 5, wherein, the first analog-to-digital conversion circuit is connected to the multiplicative-artifact analysis module; the first analog-to-digital conversion circuit is configured to output the first potential-difference signal, for which the analog-to-digital conversion is implemented, to the multiplicative-artifact analysis module.

7. The human-body-signal collection device according to claim 2, further comprising a first sensing device, wherein:
the first sensing device is connected to the first signal processing device; the first sensing device is configured to detect a first motion signal of the first electrode and output the first motion signal to the first signal processing device; and the first signal processing device is configured to judge whether the first signal processing device works or not according to the first motion signal.

8. The human-body-signal collection device according to claim 7, wherein when the first motion signal is greater than a preset value, the first signal processing device works.

9. The human-body-signal collection device according to claim 1, further comprising:
a third electrode, attached to the torso of the human body, and configured to output a third signal;
a second collection device, connected to the second electrode and the third electrode, respectively, and configured to collect the second signal and the third signal, and generate a second potential-difference signal according to the second signal and the third signal; and
a second feedback device, connected to the second collection device and the adjusting and generating device, respectively, and configured to collect the reference signal and the second potential-difference signal, and configured to generate a second feedback signal according to the reference signal and the second potential-difference signal, and further configured to use the second feedback signal to compensate for the second potential-difference signal.

10. The human-body-signal collection device according to claim 9, further comprising:
a switch, wherein the switch comprises a signal input terminal, a first output terminal, and a second output terminal; the signal input terminal is connected to an output terminal of the adjusting and generating device; the first output terminal is connected to the first electrode; and the second output terminal is connected to the third electrode.

11. The human-body-signal collection device according to claim 9, further comprising:
a central control device, wherein the central control device is connected to the first feedback device and the second feedback device, respectively; and the central control device is configured to collect the first potential-difference signal and the second potential-difference signal, in which artifacts are eliminated, and obtain detected signals based on the first potential-difference signal and the second potential-difference signal.

12. The human-body-signal collection device according to claim 9, wherein, the human-body-signal collection device further comprises a power supply module; and
the power supply module comprises:
a power management module, connected to the adjusting and generating device, the first collection device, the first feedback device, the second collection device, and the second feedback device, respectively; and
a battery, connected to the power management module.

13. The human-body-signal collection device according to claim 1, further comprising:
a third executing device, connected to the first collection device, to offset capacitance between the human body and a reference ground.

14. The human-body-signal collection device according to claim 13, wherein the reference electrode, the first electrode and the second electrode are wet electrodes.

15. The human-body-signal collection device according to claim 13, wherein the first collection device further comprises:
a first amplifier, wherein a first input terminal of the first amplifier is connected to the first electrode, a second input terminal of the first amplifier is connected to the second electrode, and the first amplifier is configured to collect the first signal and the second signal, and generate the first potential-difference signal according to the first signal and the second signal;
a first gain amplifier, connected to an output terminal of the first amplifier, and configured to implement a gain for the first potential-difference signal;
a first filter, connected to the first gain amplifier, and configured to implement a filtering for the first potential-difference signal, for which the gain has been implemented; and
a first analog-to-digital conversion circuit, connected to the first filter, the first executing device, and the first signal processing device, respectively, and configured to implement an analog-to-digital conversion for the first potential-difference signal, for which the filtering has been implemented.

16. The human-body-signal collection device according to claim 15, wherein:
the first feedback signal comprises a third sub-feedback signal;
a first terminal of the third executing device is connected between the first electrode and the first input terminal of the first amplifier; a second terminal of the third executing device is connected between the output terminal of the first amplifier and the input terminal of the first gain amplifier; a control terminal of the third executing device is connected to the first signal processing device;
the first signal processing device is further configured to obtain the third sub-feedback signal according to the reference signal and the first reference drift signal, and output the third sub-feedback signal to the control terminal of the third executing device;
the control terminal of the third executing device is configured to adjust a capacitance value of the third executing device according to the third sub-feedback signal.

17. The human-body-signal collection device according to claim 16,
wherein the third executing device is connected in parallel with the first amplifier; and the third executing device is configured to adjust the capacitance value of the third executing device according to the third sub-feedback signal.

18. The human-body-signal collection device according to claim 17, wherein the third executing device is a variable capacitor.

19. The human-body-signal collection device according to claim 18, further comprising a first bias adjustment device connected between the first terminal of the third executing device and the first electrode.

20. The human-body-signal collection device according to claim 19, wherein, a signal terminal of the first bias adjustment device is connected to the first feedback device; the first feedback device is configured to generate a voltage adjustment signal according to the reference signal and the first potential-difference signal, and configured to output the voltage adjustment signal to the first bias adjustment device; the first bias adjustment device is configured to adjust a bias voltage according to the voltage adjustment signal, to realize a compensation for a DC bias voltage at the input port of the first collection device.
